(19) United States
(12) Patent Application Publication
(10) Pub. No.: US 2003/0181845 A1
(43) Pub. Date: Sep. 25, 2003

(54) Preparation and delivery of healthcare services utilizing electrolytic medicament

(76) Inventor: Kevin R. Orton, San Clemente, CA (US)

Correspondence Address: Kevin R. Orton, 257 Avenida Lobeiro, Unit G, San Clemente, CA, 92672, US

(21) Appl. No.: 10/102,498

(22) Filed: Mar. 21, 2002

(51) Int. Cl. A61N 1/30

(52) U.S. Cl. 604/20

(57) ABSTRACT

Means for delivery of healthcare services utilizing electrically active substances. Included is a machine for preparing electrically active substances, a vial, packaging and data handling means for preparation and delivery of such substances. The data handling means and machine provides for improved preparation and delivery of such substances, with more predictable dosage potencies and greater business efficiency. The vial provides for sterile handing of such substances, and reduces the chance of operator error.

### BACKGROUND OF THE INVENTION

This invention relates generally to the health care industry, and generally providing improved health care to individuals in preparation of electrically active substances for use as medicaments.

There are numerous injectable medicaments in use today, most all generally chemical based. Some emerging types of drugs are prepared with an electrical process. Such electrically prepared drugs offer a new avenue of advancement in a traditional drug field. This invention provides means whereby certain types of electrical medicaments may be more easily and more safely and effectively prepared and administered than present techniques provide. It allows for easier, better access to electrically prepared medications than present techniques.

One aspect of the invention is geared toward preparation and administration of electrically prepared substances. Electrically active prepared substances may be used for their medicinal qualities. The substances are typically prepared for use with the application of an electrical current applied to an electrolytic substance. The substance is then used as a medicament for injection or application to a recipient. The electrical signal changes a physical property of the fluid and provides medicinal qualities. The substance is then used as a medicament.

An electrical signal generator is generally used to prepare the substance for injection or application. Additional information may be found in co-pending application Ser. No. 09/289,409. The electrically prepared active substance is usually only effective for a limited period of time after the application of the electrical signal, therefore the electrical signal generator is generally located at the site of use.

One problem of locating the signal generator at the site of use, is maintaining the quality control of the drug or treatment administration. For example, the manufacturer of the signal generation hardware device may build the activator device and program it with certain signal generator electrical characteristics for preparing the substance. The manufacturer may also provide a fluid substance or drug for use in the manufacturer's activator machine. If either the electrical characteristics of the activator machine or the chemical composition of the activated substance is altered before or during preparation, it can change the treatment dosage and/or treatment properties of the active treatment substance. This can have undesired and unintended effects on the medication and patient.

Another problem that occurs is maintaining purity of the fluid prior to injection. Present techniques too easily permit contamination of the fluid. Thus a better technique is desired. This invention provides such a means.

Another problem that is encountered is that of lot expiration. This happens where the shelf life of the medication expires before the medication is all used up. Normally, the medication should be thrown away, or discarded or destroyed. However, it may happen in the course of normal use that expired medication is improperly or accidentally given to a recipient. This can cause reduced effectiveness, unstable or unknown dosage potency, improper treatment or even harm to the patient. It is desirable to prevent this from occurring. This invention provides such a means. Another problem is one that occurs in the distribution of the medicament. Often times regulations require that the lot or batch number of every vial produced be tracked and recorded throughout the distribution process. Each time the medicament changes hands between the manufacturer, the distributor, and the health care practitioner, the lot number is recorded. Then if a bad lot or batch is found, other users possessing the same lot or batch may be located and notified, and the defective lot or batch may recalled. This may be time consuming, and somewhat error prone. Thus it is desirable to provide a method that provides easy and economical administration, yet equal or better reliability. This invention provides such a means.

Another problem that occurs is improper adjustment of dials on the signal generator. For example, a medicament of formula “A” may require a 50 Khz signal be applied to it. The literature accompanying the medicament may specify a frequency of 50 Khz be used to prepare the medicament. When the operator begins preparation of the medicament, the operator may inadvertently adjust the signal generator to a frequency other than 50 Khz. The operator may for example inadvertently set the signal generator to a frequency of 55 Khz, or some other improper frequency. This can have very undesirable effects on medicament preparation. For example, excessive pressure may form in the vial. Thus a better technique is desired. This invention provides such a means.

The electrical signal generator may have an adjustable output waveform. It is often desirable that an adjustable waveform be used. Such adjustable waveforms allow the practitioner to provide a certain limited range of adjustments when preparing electrically prepared medications, so as to allow a variation in treatment modalities.

However, when adjustable signal waveforms are permitted by the user, there may exist the possibility of undesired consequences. For example, in the case of a sealed vial, if an improper dc bias current is used, the vial or seal may burst as the result of an electrolytic pressure build up in the vial.

One reason electrically prepared drugs have not found widespread use is because the preparation process causes unpredictable or unstable dosage strengths, and therefore erratic potency levels. This can cause erratic and inconsistent treatment results, reducing the credibility and practice of treatments. Another problem is setting the dials and controls of the signal generator so as to obtain proper results. If the settings are not properly matched to the fluid in use, or are bumped of moved improper results can occur. This can cause poor quality health care. Stable and predictable dosage potencies are desirable in order to provide quality treatments. This invention provides means whereby stable dosages of electrically prepared pharmaceuticals may be prepared, with more consistent, predictable results, and less chance of operator error. Another problem which may occur is if the correct fluid substance is not used with the proper signal generator waveform. For example, some fluids may be designed for use with a 45 Khz waveform, while others may use a 50 Khz waveform. A practitioner may posses some fluid designed for use with a 50 Khz square waveform, while certain signal generators may only produce a 45 Khz wave. The user may be tempted to use the improper settings, and not obtain the full and proper benefit of the fluid. This can cause undesired results. Thus it is desirable to provide a means whereby the user may be permitted to use a variety of different fluid treatments, without danger or risk of setting the incorrect waveform for each fluid. This invention provides such a means.

### BRIEF DESCRIPTION OF THE DRAWINGS

FIG. 1 shows a novel medicament vial suitable for practicing the invention.

FIG. 2 shows a signal generating machine.

FIG. 3 shows a cutaway view of a portion of the signal generator.

FIG. 4 is a flowchart showing how data is prepared.

FIG. 5 is a block diagram showing how various portions including data handling of the system work together.

FIG. 6 is a diagram further illustrating aspects of the system.

### DETAILED DESCRIPTION OF THE DRAWINGS

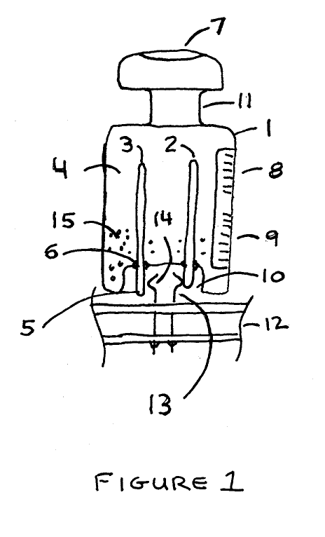

In one aspect of the invention, a vial is provided. In the case of electrically prepared medicaments the vial contains several advantageous and unique aspects. The vial FIG. 1 has a housing 1. Included in the housing are electrode elements 2 and 3 that pass from the inside 4 to the outside 5 of the vial. The inner portion 3 and the outer portion 5 of the electrode is sealed 6 against the housing 1 in a sterile manner. Means comprising an access port 7 is provided. The access port may consist of a soft pierceable rubber portion, through which a hypodermic needle may be inserted, or other means to access the contents of the vial, preferably in a sterile manner.

The vial may include a number code 8 or multiple number codes 9. The number code may include serialization, and authentication data. The number code may be placed on the side of the vial, or any convenient location. The number code may take any of many types of human or machine or electronically readable data formats, such as bar codes, alpha-numeric digits, eeprom memory devices, and the like.

In the preferred embodiment, the vial is round and configured so as to be processable by automatic bottling and filling equipment. In one aspect of the invention, the lower portion of the vial consists of a hollow cavity 10 in which electrodes 2 and 3 are positioned. The electrodes are recessed inside the hollow cavity, protecting the electrodes from damage, and providing a flush bottom surface that does not impeded movement of the vial on conveyorized machinery. Further, the vial may include a neck portion 11 to facilitate automated handling. A preparation machine 12 includes contacts 13 and 14 for making an electrical connection to electrodes 2 and 3, thereby permitting the passing of electrical current through the contents 15 of the vial. The hollow cavity 10 or other portion may include a keyed portion that facilitates alignment of the electrodes 2 and 3 with the contacts 13 and 14. The keyed portion may consist of a notch, flat spot, groove, or the like, preferably located on the lower portion of the vial.

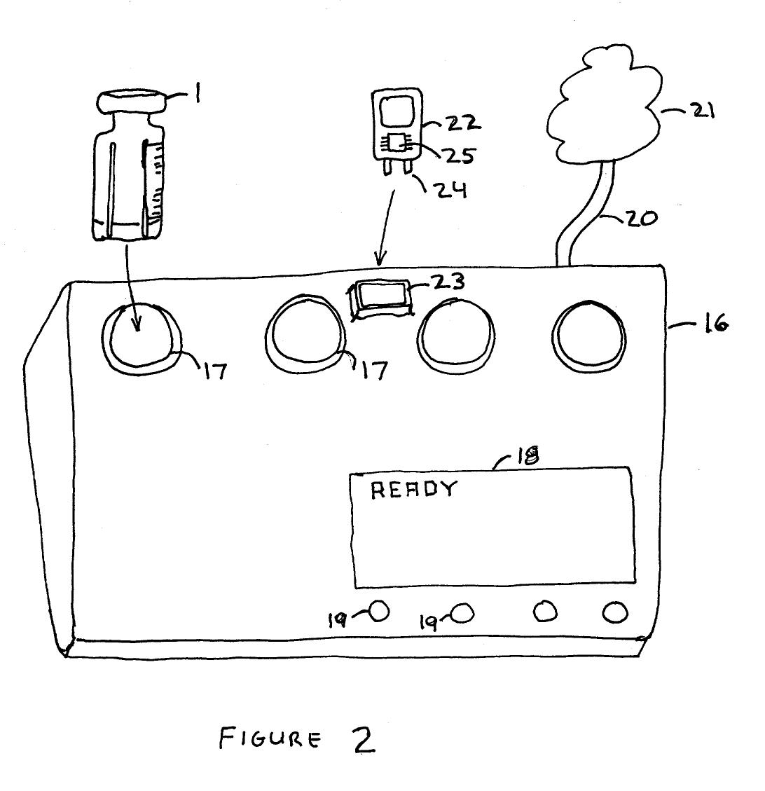

FIG. 2 shows a signal generating machine 16 for preparation of fluid substances in the vial 1. According to one aspect of the invention, the machine may include a housing 16 and one or more chambers 17 fitted to accommodate the vial 1. The chambers hold the vials, and facilitate transfer of information on or in the vial with the machine. The machine may also include readout display means 18, and control buttons 19. The display 18 and control buttons 19 facilitate interface with an operator. The operator may be able to enter such commands as the time of day, user identification, machine ownership data, setup and operating commands and parameters, special commands for the preparation of medicaments in the vial, and the like. The machine may also include a power supply and a modem, or other linking means 20 to facilitate communication with or over a network 21 and other accompaniments. The machine may operate under microprocessor control, or be linked to a microprocessor or other computing device. The machine may include an elapsed time timer that determines when the medicament is ready for use, and means to alert the user. Furthermore, the machine may include a patient database of user and medication preparation activity, or be linked to such a database. The machine may be leased to the user, or sold on an outright basis.

Such a patient database may record for example the name of the patient, the type of medication given, the electrical parameters, such as frequency and current used to prepare the medication, the lot number, the operator identification, name of the physician, ect. The database provides an easy and convenient means of keeping track of treatment progress.

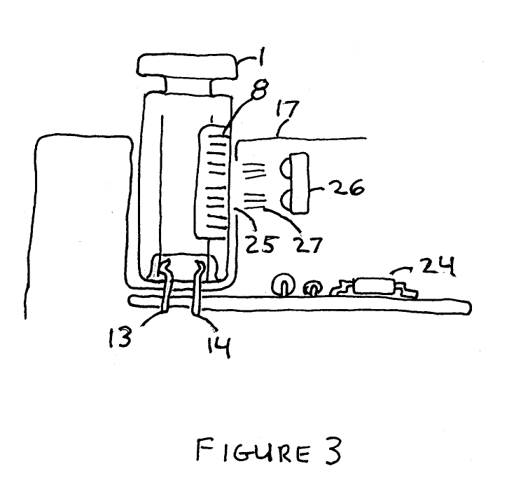

FIG. 3 shows a cutaway view of the chamber of one embodiment of the machine of FIG. 2. The chamber 17 has contacts 13 and 14 that form an electrical connection between the vial 1 and signal generating circuitry 24. According to one aspect of the invention, an opening 25 in the signal generator apparatus is preferably configured with a sensing means to detect the presence of the vial in the generator, as well as the coding placed on or attached to the vial. Such sensor means 26 may be fitted to the chamber area 17. Such sensor means is in communication with the data 8 on the vial 1. The sensing means may include infra-red light waves 27 and detectors, eeprom reading circuits, electrical connections, bar code scanner readers or other means such as light beams, photocells, and the like to transfer data stored on the vial to the machine. The data is transferred from the vial 1 to the signal generation machine 16 circuitry 24. Generally, the data is preferably in digital format.

In use, the user obtains the vial though whatever distribution channel they prefer. The user may use multiple suppliers. When the user is ready to use the vial, the user places the vial into the holding chamber opening provided by he signal generator apparatus. A light beam shines on the bar coding, and a photo-detector detects the reflected light. The signal is amplified and the waveform is cleaned up to produce two digital states, 1 and 0, corresponding to reflective and non-reflective portions of the bar code. The machine is able to read the data associated with the vial. The machine reads the data, usually in digital format, and uses the data to configure the operating parameters of the signal generator of the machine. The data reading apparatus is in communication with the signal generating circuitry. The data reading step is thus interlocked with the signal application step, thereby preventing incorrect use of the system by inserting the wrong vial data.

Alternately, the data need not be on the vial at all. A small token device 22 FIG. 2 may accompany the vial, but be separate from the vial. In this case, the token device may be inserted into an opening 23 of the machine, and supply data to the machine with electrical contacts 24, bar codes, or the like. The token device may include an eeprom 25 or other memory device. The token device may also consist of a magnetic or paper card or other device that holds data. The separate data device may contain data for more than one vial at a time, and may be inserted into the machine ahead of time, prior to using the medicaments. Token-like data may alternately be entered manually by the user using buttons or keys. (FIG. 2, 19.)

To prevent defeating the system, the sensor 26 is preferably active while the vial electrodes 13 and 14 are in contact with the signal generator circuitry 24. The code sensor is preferably placed along side of the well portion that retains the vial. The sensor may work with the signal generator electrical circuitry to detect if the vial is removed from the machine after it has been read. If the vial is removed or replaced after the read scan verification process, the signal generator may turn off and give an error signal. The vial may then need to be re-scanned and re-verified again, thus preventing improper operation of the machine.

In one aspect of the invention, a sealed vial is used to hold the medicament. The seal is placed on the vial after the vial is filled in a sterile manner. The vial may be transported of stored with the seal and the electrodes intact. The vial has electrodes and wire connections built into the vial. Wires from a signal generator connect to the electrodes in the vial, and allow current to flow through the fluid substance without opening the vial.

One problem that occurs is the accidental or improper use of expired medicaments. Many medicaments must be used within a certain period of time, or they can lose their potency or become unstable. To this end, often times an expiration date is printed on the medicament. The user checks the expiration date before using the medication, and if the medicament is found expired, the medicament is discarded before use. Many times however, users may forget to check the expiration date before use. This can result in the accidental use of expired medicaments, which can reduce the effectiveness of the treatment for the patient, or cause undesirable effects. Other times the user may check the expiration code and find the medicament is expired. However, though the user knows the medicament is expired, the user may be tempted to use the medicament anyway, to possibly detrimental effects. Thus it is desirable to provide a way to reduce or eliminate the accidental or intentional use of expired medicaments.

In one aspect of the invention, data placed on the vial includes information as to the expiration date of the medicament in the vial. The expiration date is read from the vial when the vial is placed in the machine and the scanning circuitry activates. The data is then processed, and compared to reference date data in the machine. If the expiration date found in the data on the vial is found to be past the current date information in the machine, the machine circuitry terminates activation preparation of the fluid in the vial by turning off the signal generating portion of circuitry. Thus the chance of inadvertent or improper use of the fluid is greatly reduced.

Another problem that occurs is assuring proper preparation of the medicament. Different medicaments may require different electrical signals to be applied in order to properly prepare them. If a user applies an incorrect electrical signal, the medicament may be improperly prepared, and it may be difficult to find the error if a problem should occur. In one aspect of the invention, configuration data is included in the data on the vial. The data associated with the vial is transferred to the machine preparing the substance in the vial. The configuration data may be linked to data in a central server, or the configuration data may be directly applied to or with the vial. The configuration data on the vial is readable by sensors or data paths in communication with the signal-generating machine and vial data.

For example, in one novel aspect, the vial may contain a product code data value of “1”. A “1” may indicate that the vial contains 2 milliliters of a certain 1% NaCl solution, and that a frequency of 50 Khz at a voltage of 35 volts is to be applied to the vial for a period of 8 hours to properly prepare it. The signal generator machine reads the value of “1” from the vial, checks with the central server database and determines what the correct operating parameters are, and sets the output circuitry of the signal generator to produce an output voltage of 35 volts at 50 Khz. The medication is then prepared. The configuration or setup data is preferably transferred in digital format. Different encoding schemes for the data transfer may be used, such as ASCII, binary, or custom data formats, and still fall within the scope of the invention. The voltage and frequency or other configuration operating parameter values may be suggested settings that the machine user can change or override, or they may be fixed such that the user can not alter the values. Different vials with different fluid solutions may have different suggested operating parameter values, depending on what treatments are being performed. The signal-generating machine reads the vial data, and sets signal generator operating parameters accordingly. Thus the chance for operator error is greatly reduced.

When the vial is loaded into the machine, the scanner or reader on the machine reads the setup code on the vial. The voltage, current, frequency, or other parameters specified on the vial are loaded into the memory of the machine. The parameters may be specified directly, or they may be coded to match a look-up table in the machine that has all the parameters associated with a certain profile. The memory may be used by the signal generating mechanism to adjust the bias voltage, reference voltage, registers or machine cycle steps in a digital oscillator or other means so as to provide the desired output signal waveform to the vial. If a look-up table is used to supply the details of a certain set of parameters, then the look-up table may be updated occasionally with data from the server database, so as to allow changes in operating parameters after the machine is delivered to the field.

Normally, each batch of medication requires a lot number. The lot number serves to provide traceability in case the lot batch should later be found to be bad, and require recall. The manufacturer records the lot number of each and every vial or dose of medication that is prepared on the medication. The medication is then conventionally distributed through third parties to end users. Each time the medication changes hands, the lot number of each vial must be recorded. Thus the lot number may need to be recorded numerous times before reaching the end user. If the medication lot should later be found to be bad, the lot number of the bad batch may be determined. The manufacturer then contacts all parties to whom bad medication was delivered. Each of these parties then contacts all parties to whom bad medication is delivered, until each dose of bad medication is located and retrieved. This can be time consuming and error prone. Thus a better way is desired.

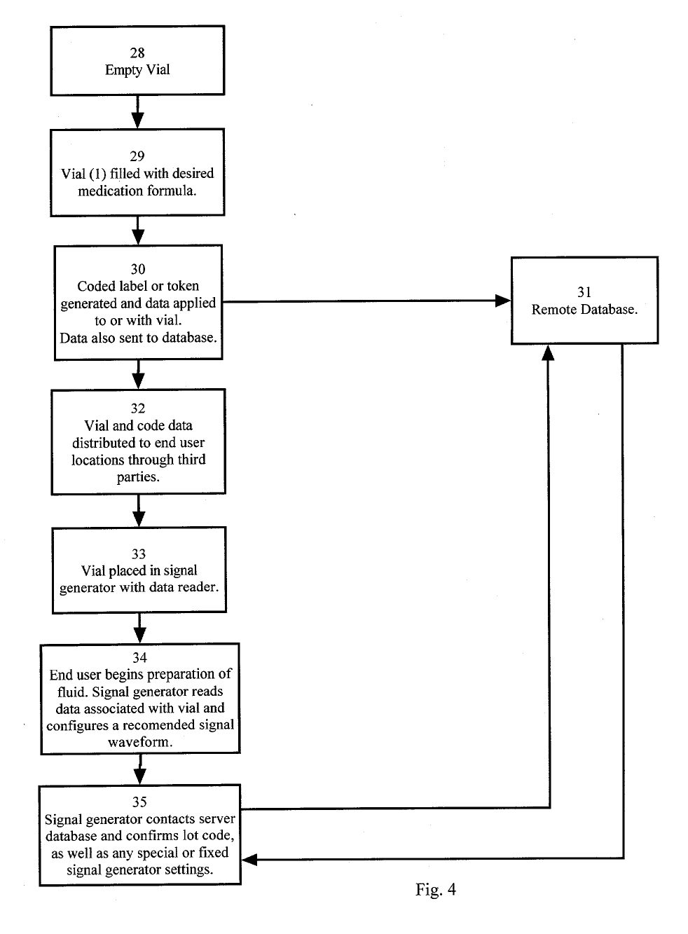

FIG. 4 is a flowchart showing how the vile data is prepared and used. When the vial is filled 29, data is encoded on the vial with the type of fluid being put in the vial. This is done preferably at the same site and same time the vial is filled. An authentication code and serial number 30 are also preferably generated and also placed on the vial. The authentication code is preferably a randomly generated or difficult to guess number. At preferably the same time, a record copy of the data placed on the vial is placed and stored in a database in a secure server 31. If a serial number and authentication code is used, these numbers will generally be unique to each vial produced. After filling the vial may then be freely distributed and redistributed through third party channels to remote locations. When the end user receives the vial, the end user places the vial in the signal generating machine chamber 33 (also FIGS. 3-17) or otherwise transfers data associated with the vial to the signal generating machine. The machine sensor reads the data from the vial, and configures the signal generating machine to the proper parameters 34. The signal generator machine may also then open an electronic communication channel from the signal generator machine to a central server database 31. The electronic communications channel may consist of a modem, uart, serial port, or other hardware device. The communication channel may consist of telephone wires, the internet, a cellular network, fiber-optic cabling or other similar means. The signal generator machine takes the data from the vial and compares it to reference data supplied by the server 31. Using the data on the vial, the server assigns recommended operating parameters to the signal generator for the signal generator to use.

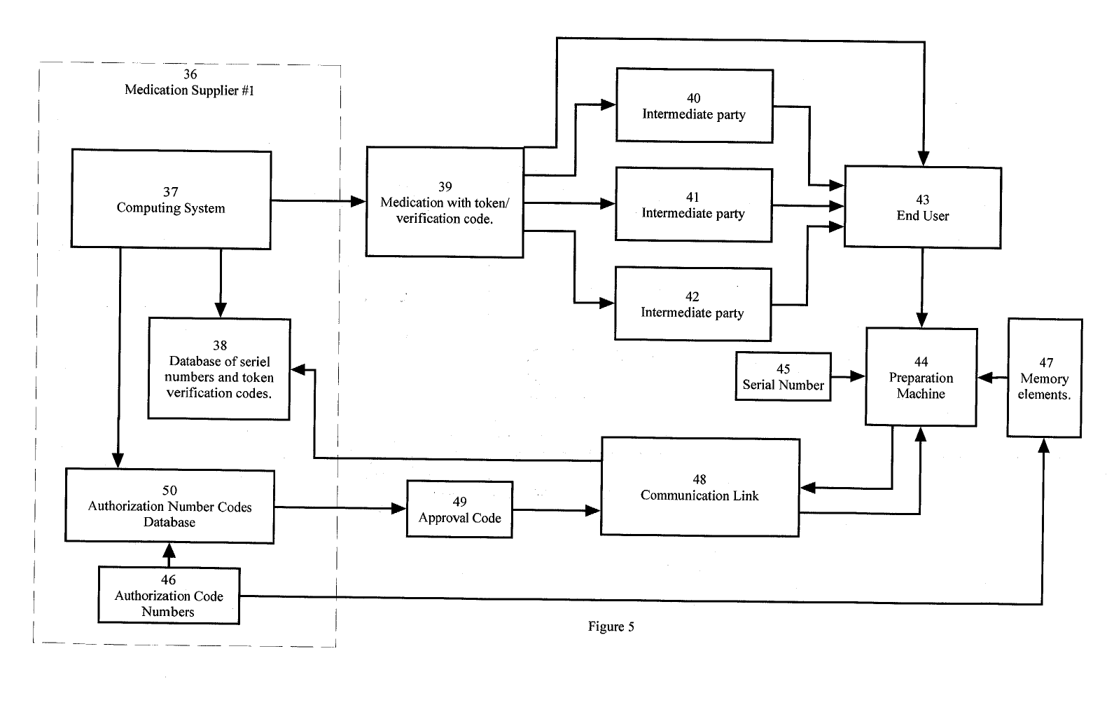

FIG. 5 is a block diagram showing additional aspects of a novel configuration and verification process when and how the medication is distributed. The medication manufacturer 36 prepares the medication. A computing system 37 may be used as an aid to preparing the medication with number codes. When preparing the medication, the manufacturer provides a token, or a number code along with the medication 39. A record of the number code for each vial is saved in a database 38. The medication with number code 39 is then distributed to one or more intermediate parties 40, 41, 42, or directly to end users 43. There is no need to track or trace or record transfers or shipments or lot numbers between the manufacturer, intermediate parties, and end users. The end user then prepares the medication for use with the aid of the preparation machine 44. The preparation machine may include a serial number 45. The preparation machine then connects to the database of the manufacturer 38, using communication link 48. The preparation machine verifies the medication is okay to administer by comparing the data on the vial with the data in the database. The verification process may also confirm that the lot has not exceeded it's expiration date, and that the lot has not been recalled or cancelled. If such an expiration or recall is issued, the database 38 is updated, and thereafter any attempts to use expired or bad medication is blocked during the authorization process. Multiple medication suppliers 36, each with their own independent database, may all use the same preparation machine 44. In this case, supplier data may be included with the number codes, so as to facilitate the preparation machine 44 with automatically connecting to the correct database 38.

If an end user 43 should try to use medication 39 with an improper or unrecognized number code, or try to improperly re-use the same medication twice, preparation machine 44 may be configured to reject the medication. Likewise, if the medication is expired, or has had a recall or bad lot number, the preparation machine may reject the medication. If the medication is a conventional medication, the machine may issue a warning tone, flash lights, or otherwise inform the user that the medication should not be used. If the medication is an electrically prepared medication, the machine may halt or cancel the electrical preparation process before the medication becomes ready to use, thus preventing improper use of expired or bad medication. Thus the system eliminates the need to track lot numbers throughout the distribution process. This arrangement also provides greater business efficiency.

The system may also be used in the case of recalls. Recalls may occur if a lot of medication is found to be bad after it has been shipped to the end user. In this case, the end user may not even be aware that the lot being used is bad, defective, or being recalled. In this case, potential harm to the patient can occur from use of the bad substance. It is desirable to prevent this from happening. This system provides a means whereby the user may be protected against use of batches or lots of substance that are bad.

If a lot or batch of substance is found to be bad, a notation is placed in the server database that such batch is bad. The notation may be placed in the server database through manual or automated means. The notation remains in the database until such time as the end user may try to use the bad lot. When the user tries to use the bad lot, the user machinery reads the code data from the vial of substance. The user machine then contacts the server database maintained by the manufacturer or other party controlling use of the substance and obtains status information from the manufacturer database. The server database is generally off-site from the end user. The user machine makes contact with the server through telephone lines, the internet, wireless transmission, or other means. The server checks the server database and verifies that the serial number or other code supplied by the user machine is recognized as belonging to the vial provider, and that the verification code on the vial is the proper one associated with that serial number. The server may also check the server database to see if there is any expiration or bad lot warnings on the vial in question. If there is a bad lot warning, or other warning, the server may notify the user hardware that the vial is bad, and should not be used. The user machine will then halt processing on the vial, and the substance will not become electrically active. If the serial number and verification code match the data stored in the database, and there are no expiration or bad lot warnings, the server may issue an approval code to the user machine. The approval code functions as a command that allows the activation process on the user machine to begin. If the verification code is not found to be associated with the serial number in the database, the information provided from the vial may be considered to be improper. In this case, the server does not issue an approval code to the user machine, and the user machine does not turn on the electrical signal used to activate the substance in the vial.

Each user machine preferably has a unique identification or serial number associated with it. The identification number is logged in a central database at the time the machine is manufactured. Along with the identification number, various randomly generated approval codes may be stored in the memory of the user machine. These approval codes are also recorded in a database, and linked with the machine identification serial number.

The current is applied via generally an alternating current waveform from a signal generator. The current flow is measured by the machine with an amp sensing circuit while the vial is connected to the machine and the current is flowing. The amp sensing circuit measures the current flowing to the electrodes in the vial. There may be different size of types of vials to be used with the machine. Each vial size of type may contain different formulations of fluid substance, and may require different voltages or currents to be applied. The machine includes stored data tables relating to the proper amount of current that should flow for the vial size and fluid type in question.

In another aspect of the invention, the system uses tamper resistant authorization signals. When a vial or token is inserted in the machine, and the machine verifies that the token is authentic with the database, the host computer 37 FIG. 5 will send a signal 49 to the machine 44 that is okay to proceed with the preparation of the medicament. This is the authorization code. However, the potential exists for a user to intercept and generate a counterfeit authorization code, signaling the machine to proceed with the preparation, even when the vial data does not match the database data. In order to prevent this action, an encryption system may be used. However, encryption systems generally require large amounts of processing power, and further, may be broken if the key is discovered. Therefore a novel approach is used. The machine 44 includes a set of numbers 46 that are programmed into memory elements 47 of the machine. The numbers are programmed during the manufacturing of the machine, and a copy of the numbers is saved in a database 50. The end user does not set, adjust, or program the numbers. The numbers 46 are generally random, and form no specific pattern. The memory elements 47 are generally part of the machine 44 and retain their memory even when the machine is not in use. The memory elements 47 are themselves numbered or indexed so that certain memory locations may be addressed. The value of the numbers 46 are randomly generated and a copy is recorded in a database 50 of authorization codes when the memory element 47 is programmed initially. Then when the machine 44 is placed in service, and a valid token is received, the computing system 37 checks the database 50 and the identification serial number 45 of machine 44, and locates the address table of known authorization codes that correspond to the serial number of that machine. Different machines 44 generally have different serial numbers 45, and different authorization code tables. When a good vial is detected in the machine, the computing system 37 may send a known good authorization code to machine 44. The machine compares the authorization code supplied by computing system 37 and database 50 against it's own table of codes in the memory elements 47, and if a match is found in the specified address, the machine proceeds with the preparation process. When comparing codes, the machine will generally check a memory location address in the memory elements 47 and the contents of the memory address, to verify they match the data supplied by the database 50. Generally, the contents of the memory address 47 need never be transmitted outside of the machine 44, thereby providing increased security. Each time a new authorization code is required, the system may use a different memory address to compare, thus blocking attempts to recognize and counterfeit a certain pattern. Hundreds or thousands or more of authorization codes may be programmed into the memory elements 47 for a modest cost. A hacker would have to generate hundreds or thousands of legitimate authorizations in order to generate a duplicate authorization code table, thus making counterfeiting unfeasible. The memory element containing the authorization code table in the machine 44 may be an eeprom device or some other device. The eeprom may physically reside inside a micro controller chip in the machine, making it difficult to break into the memory table 47 without also damaging the machine. As a further security measure, the machine may generate a random selection of which memory element address location 47 to use the comparison data from for each authorization process, making counterfeiting by characterization of the system very difficult. Preferably, each machine has it's own set of memory address values.

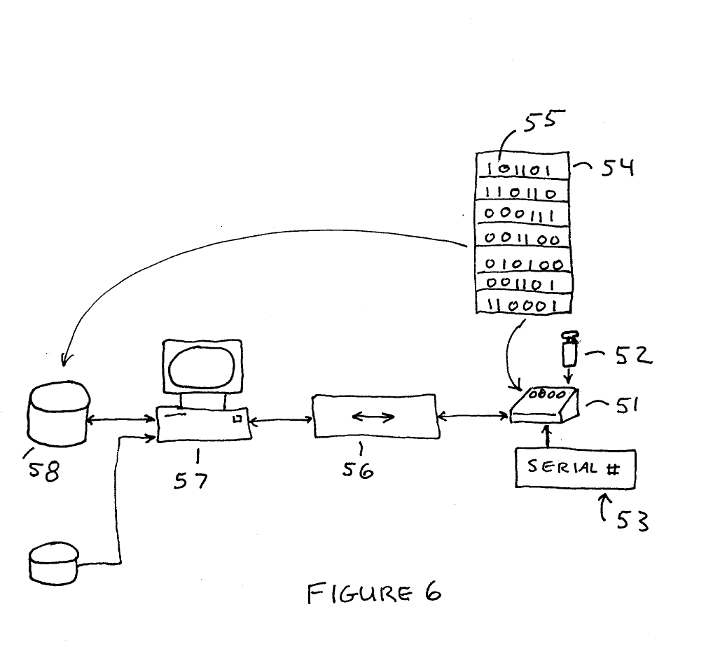

An alternate rendition of FIG. 5 is partially shown in FIG. 6, and includes a machine, a vial 52, a serial number 53, memory elements 54, code numbers 55, a communication link 56, a host computer 57, and a database 58.

While the tamper resistant authorization code of the system may be used with machines for preparation of medicaments, such an authorization system may alternately be used on other applications as well, such a utility meters and subscription services. The machine may either form an electronic connection 48, or use a keypad or other means whereby the user manually enters authorization codes provided by the operator of the database.

The vial is preferably pre-filled under sterile conditions, and then transported to the site of use. Numerous advantages are obtained in this manner. Pre-filling the vial at the factory allows controlled filling conditions, which provides much better sterile integrity than can be obtained by sterilizing the fluid in the field. Automated equipment is available which allows the vial to be filled in a sterile manner with high reliably. The vial may then be electrically prepared in the field, without compromising the sterile seal until the time of use.

Thus the system provides for controlled and traceable delivery and preparation of electrically prepared medicaments, with reduced chance of operator error, and higher quality. While exemplary examples are shown, numerous variations may be made and still fall within the spirit and scope of the invention.

What is claimed is:

1) A machine for preparing electrically active medicaments, said machine including a signal generator and means for connecting said generated signal to a vial of medicament.

2) A machine as in claim 1, further comprising a sensing means for reading data from the vial.

3) The machine of claim 2, wherein data associated said vial is compared with data at an off-site database.

4) The method of claim 3, wherein the result of said data comparison is used to control said signal generating means.

5) A vial of medicament for preparation with electrical signals comprising a housing, a sterile seal, an access port, and at least one electrode in contact with fluid inside the vial, said electrode also including means for electrical connection to a signal generator outside of said housing.

6) A system as in claim 3, wherein said data in said database is used to set operating parameters of said machine according to data supplied with the vial.

7) A machine for reading data associated with a dose of medicament, wherein read data associated said medicament is compared with data at an off-site database, wherein said comparison data is used to signal the machine operator that said medicament may be unfit for use.

8) A method as in claim 3, wherein said comparison data is used to disable said signal generating means if said comparison data indicates the contents of said vial is expired.

9) A method as in claim 3, wherein said comparison data is used to disable said signal generating means if said comparison data indicates the contents of said vial may be unfit for use.

10) A method as in claim 3, wherein said comparison data is used to modify the output of said signal generating means depending on contents of said data.

11) Providing a vial which is sealed in a sterile manner with an electrolytic substance inside the vial, said vial containing electrode elements which are in contact with said electrolytic substance inside the vial, said electrode elements also extending to the outside area of the vial, said vial being pre-filled at one location, said vial then being delivered to another location, at which said second location an electrical signal generating apparatus is applied to the electrodes of said vial, said contents of said vial then being removed from said vial and administered to a recipient.

12) A system for authorizing use of services, said system comprising a database, an enabling machine, and a data connection, said enabling machine being programmed with a confidential set of authorization codes prior to distribution to an end user, a copy of said confidential authorization codes being kept in a secure database, said machine making contact with said database when use of services are desired, said database supplying one of the pre-arranged authorization codes to said machine, and; said machine enabling services if a comparison of said authorization code in said database matches the prearranged authorization code in said machine.

13) A system as in 12, wherein multiple authorization codes are stored in memory elements of said machine and said database, each authorization code being individually addressable, the address of the authorization code to use when making comparisons of data in said database and said machine changing periodically.
